(19) United States
(12) Patent Application Publication
(10) Pub. No.: US 2024/0165134 A1
(43) Pub. Date: May 23, 2024

(54) COMPOSITIONS AND METHODS FOR TREATING BLADDER CONDITIONS

(71) Applicants: HADASIT MEDICAL RESEARCH SERVICES & DEVELOPMENT COMPANY LTD., Jerusalem (IL); BUZZELET DEVELOPMENT AND TECHNOLOGIES LTD., OR AKIVA (IL)

(72) Inventors: Guy HIDAS, Reut, IL (US); Noa RAZ, Gizo (IL); Elyad DAVIDSON, Jerusalem (IL); Aharon EYAL, Jerusalem (IL)

(21) Appl. No.: 18/272,638

(22) PCT Filed: Jan. 17, 2022
(86) PCT No.: PCT/IB2022/050341

§ 371 Date: Jul. 17, 2023

Related U.S. Application Data
(60) Provisional application No. 63/138,540, filed on Jan. 18, 2021.

(51) Int. Cl. A61K 31/00; A61K 9/00; A61K 9/107; A61K 45/06

(52) U.S. Cl. CPC A61K 31/658; A61K 9/0034; A61K 9/107; A61K 45/06

(57) ABSTRACT

Provided are methods for treating a bladder condition in a subject in need thereof by administering to said subject a therapeutically effective dose of a composition comprising at least one cannabinoid and a carrier. Also provided are cannabinoid composition comprising at least one cannabinoid, a carrier and at least one drug selected from the group consisting of an anticholinergic drug and an adrenergic drug and uses of such compositions for treating a bladder condition.

### CROSS-REFERENCE TO RELATED APPLICATIONS

The present application gains priority from U.S. Provisional Patent Application Ser. No. 63/138,540 filed Jan. 18, 2021 which is incorporated by reference as if fully set-forth herein.

### FIELD OF THE INVENTION

The field of art to which this invention generally pertains is treatment of bladder conditions, and more specifically to methods of treatment of bladder conditions by administering a composition comprising at least one cannabinoid and a carrier.

### BACKGROUND OF THE INVENTION

Bladder condition and/or dysfunction is an abnormal functioning of the bladder due to neurological, idiopathic, or inflammatory etiology.

Neurogenic bladder is a bladder condition caused by neurologic damage resulting from spinal cord lesion. Symptoms may include bladder overactivity (resulting in urinary frequency urgency incontinence) and in severe cases urinary tract infection and kidney function deterioration or bladder underactivity (resulting in urinary retention). Additional bladder conditions include cystitis, which comprises inflammation of the bladder, and chronic prostatitis/chronic pelvic pain syndrome, which is characterized by pelvic or perineal pain.

Known treatments include bladder self-intermittent catherization, medication and surgery.

### SUMMARY OF THE INVENTION

According to an aspect of some embodiments of the present invention, there is provided a method of treating a bladder condition in a subject in need thereof, the method comprising administering to said subject a therapeutically effective dose of a composition comprising at least one cannabinoid and a carrier.

According to an embodiment, said bladder condition is a bladder dysfunction.

According to an embodiment, said bladder dysfunction is selected from the group consisting of a neurogenic bladder dysfunction, an idiopathic bladder dysfunction, an inflectional bladder dysfunction, an overactive bladder, and combinations thereof.

According to an embodiment, said bladder condition is selected from the group consisting of interstitial cystitis, hemorrhagic cystitis, radiation cystitis, cyclophosphamide-induced cystitis, chronic prostatitis/chronic pelvic pain syndrome (CPPS) and combinations thereof.

According to an embodiment, said at least one cannabinoid is selected from the group consisting of THC and THCa and combinations thereof.

According to an embodiment, said at least one cannabinoid is selected from the group consisting of CBD and CBDa and combinations thereof.

According to an embodiment, said at least one cannabinoid comprises at least one selected from the group consisting of THC and THCa and at least one selected from the group consisting of CBD and CBDa.

According to an embodiment, said at least one cannabinoid comprises at least one selected from the group consisting of THC and THCa and at last one selected from the group consisting of CBD and CBDa, wherein a (total THC+THCa) to (total CBD+CBDa) mol/mol ratio is between 3:1 and 1:3.

According to an embodiment, said at least one cannabinoid comprises at least one selected from the group consisting of THC and THCa and at least one selected from the group consisting of CBD and CBDa, wherein a (total THC+THCa) to (total CBD+CBDa) mol/mol ratio is less than 0.1:1.

According to an embodiment, said at least one cannabinoid is present at a concentration of from about 1% to about 35% w/w of the total composition.

According to an embodiment, said composition further comprises at least one terpene selected from the group consisting of pinene, limonene, linalool, caryophyllene, myrcene, humulene, terpineol, nerolidol, bisabolol, geraniol, eucalyptol, borneol, sabinene, terpinolene, ocimene and combinations thereof. According to one such embodiment, a total terpenes to total cannabinoids weight/weight ratio in said composition is at least 0.05:1.

According to an embodiment, the method further comprises administering to said subject a therapeutically effective dose of at least one drug selected from the group consisting of an anticholinergic drug, a beta-3 agonist drug, botulinum toxic drug, an adrenergic drug, and combinations thereof.

According to an embodiment, said composition further comprises at least one drug selected from the group consisting of an anticholinergic drug, a beta-3 agonist drug, a botulinum toxic drug, an adrenergic drug, and combinations thereof.

According to an embodiment, said at least one drug is selected from the group consisting of Oxybutinin, Tolterodine, Trospium, Fesoterodine (Toviaz), Mirabegron (Myrbetriq), Botulinumtoxin A and combinations thereof.

According to an embodiment, said composition further comprises a surfactant.

According to an embodiment, said composition is an oil-in-water emulsion.

According to an embodiment, said cannabinoid is present in said composition in an amount of between 1 mg and 100 mg.

According to an embodiment, said administering is selected from the group consisting of intravesical administration, rectal administration, oral administration, nasal administration, sublingual administration, vaginal administration, and inhalation.

According to an embodiment, said administering is intravesical administration.

According to an embodiment, said administering uses a device that brings said cannabinoids in contact with bladder internal wall.

According to an embodiment, the method further comprises emptying the subject's bladder prior to said administering.

According to an aspect of some embodiments of the present invention, there is provided a cannabinoid composition comprising at least one cannabinoid, a carrier and at least one drug selected from the group consisting of an anticholinergic drug, a beta-3 agonist drug, a botulinum toxic drug, an adrenergic drug, and combinations thereof.

According to an embodiment, the composition further comprises a surfactant.

According to an embodiment, the composition is an oil-in-water emulsion.

According to an embodiment, the composition as disclosed herein is for use in treating a bladder condition.

### DETAILED DESCRIPTION OF THE INVENTION

The present invention relates to methods of treatment of bladder conditions by administering a composition comprising at least one cannabinoid and a carrier.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

As used herein, the term “treating” includes preventing, curing, ameliorating, mitigating, and reducing the instances or severity of a condition or a symptom thereof.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

According to an aspect of some embodiments of the present invention, there is provided a method for treating a bladder condition in a subject in need thereof, the method comprising administering to said subject a therapeutically effective dose of a composition comprising at least one cannabinoid and a carrier.

According to an embodiment, said bladder condition is a bladder dysfunction.

According to an embodiment, said bladder dysfunction is selected from the group consisting of a neurogenic bladder dysfunction, an idiopathic bladder dysfunction, an inflectional bladder dysfunction, an overactive bladder, and combinations thereof.

According to an embodiment, said bladder condition is selected from the group consisting of interstitial cystitis, hemorrhagic cystitis, radiation cystitis, cyclophosphamide-induced cystitis, chronic prostatitis/chronic pelvic pain syndrome (CPPS) and combinations thereof.

According to an embodiment, said at least one cannabinoid comprises one cannabinoid, two cannabinoids, three cannabinoids, four cannabinoids or more than four cannabinoids.

As used herein, said at least one cannabinoid is selected from the group consisting of tetrahydrocannabiniol (THC), tetrahydrocannabiniolic acid (THCa), cannabidiol (CBD), cannabidiolic acid (CBDa), cannabigerol (CBG), cannabigerolic acid (CBGa), cannabinol (CBN), cannabinolic acid (CBNa), cannabichromene (CBC), cannabichromenic acid (CBCa), cannabicyclol (CBL), cannabicycol acid (CBLa), tetrahydrocannabivarin (THCV), tetrahydrocannabivarin acid (THCVa), cannabidivarin (CBDV), cannabigerovarin acid (CBDVa) and combinations thereof.

According to an embodiment, said at least one cannabinoid is selected from the group consisting of tetrahydrocannabiniol (THC) and tetrahydrocannabiniolic acid (THCa) and combinations thereof. According to an embodiment, the cannabinoid comprises THC. According to an embodiment, the cannabinoid comprises THCa. According to an embodiment, the cannabinoid comprises both THC and THCa.

According to an embodiment, said at least one cannabinoid is selected from the group consisting of cannabidiol (CBD) and cannabidiolic acid (CBDa) and combinations thereof. According to an embodiment, the cannabinoid comprises CBD. According to an embodiment, the cannabinoid comprises CBDa. According to an embodiment, the cannabinoid comprises both CBD and CBDa.

According to an embodiment, said at least one cannabinoid comprises at least one selected from the group consisting of THC and THCa and at least one selected from the group consisting of CBD and CBDa.

According to an embodiment, said at least one cannabinoid comprises THC and CBD. According to an embodiment, said at least one cannabinoid comprises THC and CBDa. According to an embodiment, said at least one cannabinoid comprises THCa and CBD. According to an embodiment, said at least one cannabinoid comprises THCa and CBDa. According to an embodiment, said at least one cannabinoid comprises THC, THCa and CBD. According to an embodiment, said at least one cannabinoid comprises THC, THCa and CBDa. According to an embodiment, said at least one cannabinoid comprises THC, CBD and CBDa. According to an embodiment, said at least one cannabinoid comprises THCa, CBD and CBDa. According to an embodiment, said at least one cannabinoid comprises THC, THCa, CBD and CBDa.

According to an embodiment, said at least one cannabinoid comprises said at least one cannabinoid comprises at least one selected from the group consisting of THC and THCa and at last one selected from the group consisting of CBD and CBDa, wherein a (total THC+THCa) to (total CBD+CBDa) mol/mol ratio is between 5:1 and 1:5, such as 5:1, about 4.5:1, about 4:1, about 3.5:1, about 3:1, about 2.5:1, about 2:1, about 1.5:1, about 1:1, about 1:2, about 1:2.5, about 1:3, about 1:3.5, about 1:4, about 1:4.5, or 5:1.

According to an embodiment, said at least one cannabinoid comprises at least one selected from the group consisting of THC and THCa and at least one selected from the group consisting of CBD and CBDa, wherein a (total THC+THCa) to (total CBD+CBDa) mol/mol ratio is less than oda, such as less than 0.1, about 0.08, about 0.06, about 0.04, about 0.02, about 0.01, about 0.005, about 0.001 or even zero.

According to an embodiment, said at least one cannabinoid is present at a concentration of from about 1% to about 35% w/w of the total composition, such as about 1%, about 5%, about 10%, about 15%, about 20%, about 25%, about 30%, or about 35% w/w of the total composition.

According to an embodiment, the carrier is selected from the group consisting of vegetable oils, water, and aqueous solutions.

According to an embodiment, said composition further comprises at least one terpene selected from the group consisting of pinene, limonene, linalool, caryophyllene, myrcene, humulene, terpineol, nerolidol, bisabolol, geraniol, eucalyptol, borneol, sabine, terpinolene, ocimene and combinations thereof. According to some such embodiments, said composition comprises one, two, three, four, five, six, seven, eight, nine, ten or eleven terpenes from the specified group. According to some such embodiments, a weight/weight ratio of total terpenes to total cannabinoids in the composition is at least 0.05:1, such as 0.05:1, 0.1:1, 0.15:1, 1:0.2, 0.25, 0.3, 0.35, 0.4, 0.45 or 0.5.

According to an embodiment, the method further comprises administering to said subject a therapeutically effective dose of at least one drug selected from the group consisting of an anticholinergic drug, a beta-3 agonist drug, a botulinum toxic drug, an adrenergic drug, and combinations thereof. According to some such embodiments, the at least one drug is selected from the group consisting of Oxybutinin, Tolterodine, Trospium, Fesoterodine (Toviaz), Mirabegron (Myrbetriq), Botulinumtoxin A and combinations thereof. According to some such embodiments, the drug is selected from the group consisting of an anticholinergic drug and an adrenergic drug, for example oxybutinine and mirabegron. According to some such embodiments, the method comprises administering an anticholinergic drug. According to some such embodiments, the method comprises administering an adrenergic drug. According to an embodiment, the method comprises administering both an anticholinergic drug and an adrenergic drug.

According to an embodiment, the composition further comprises at least one drug selected from the group consisting of an anticholinergic drug, a beta-3 agonist drug, a botulinum toxic drug, an adrenergic drug, and combinations thereof. According to some such embodiments, the composition comprises an anticholinergic drug. According to some such embodiments, the composition comprises an adrenergic drug. According to some such embodiments, the composition comprises an anticholinergic drug and an adrenergic drug.

According to an embodiment, the anticholinergic drug is an antimuscarinic agent.

According to an embodiment, said composition further comprises a pharmaceutically acceptable a surfactant. As used herein the terms “surfactant” and “emulsifier” are used interchangeably. Examples of suitable surfactants include, without limitation, anionic surfactants (such as sodium lauryl sulfate and/or docusate sodium); cationic surfactants (such as benzalkonium chloride and/or cetylpyridinium chloride); amphoteric surfactants (such as betaines, sulfobetaine, amino acids or phospholipids); and non-ionic surfactants (such as a surfactant selected from the group consisting of re Polyoxyethylene sorbitan fatty acid esters (Polysorbate, Tween®), Polyoxyethylene 15 hydroxy stearate (Macrogol 15 hydroxy stearate, Solutol HS15®), Polyoxyethylene castor oil derivatives (Cremophor® EL, ELP, RH 40), Polyoxyethylene stearates (Myrj®), Sorbitan fatty acid esters (Span®), Polyoxyethylene alkyl ethers (Brij®), and Polyoxyethylene nonylphenol ether (Nonoxynol® and combinations thereof).

According to an embodiment, said composition is an oil-in-water emulsion.

According to an embodiment, said dose of said cannabinoid is between 1 mg and 100 mg, such as 1 mg, about 2 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or 100 mg.

According to an embodiment, said administering is selected from the group consisting of intravesical administration, rectal administration, oral administration, nasal administration, sublingual administration, vaginal administration, and inhalation.

According to some embodiments, the at least one cannabinoid and the anticholinergic drug and/or adrenergic drug are administered in a single dosage form.

According to some embodiments, the at least one cannabinoid and the anticholinergic drug and/or adrenergic drug are administered in separate dosage forms.

According to some embodiments, administering of the at least one cannabinoid and the anticholinergic drug and/or adrenergic drug in separate dosage forms is carried out independently, sequentially, simultaneously, or concomitantly, or any combination thereof. For example, the at least one cannabinoid may be administered prior to, subsequent to or simultaneously with the anticholinergic drug and/or adrenergic drug.

According to some embodiments, administering of the at least one cannabinoid and the anticholinergic drug and/or adrenergic drug in separate dosage forms independently, sequentially, simultaneously, or concomitantly, may be carried out by the same or different routes of administration. As non-limiting examples, both the at least one cannabinoid and the anticholinergic drug and/or adrenergic drug may be administered by intravesical administration or the at least one cannabinoid may be administered by intravesical administration and the anticholinergic drug and/or adrenergic drug may be administered by sublingual administration.

According to an embodiment, said administering is intravesical administration. According to some such embodiments, administration is achieved using a device such as a catheter configured to drain the bladder and apply the medication into the bladder under sterile conditions, thereby bringing said cannabinoids in contact with bladder internal wall.

According to an embodiment, the method further comprises emptying the subject's bladder prior to said administering.

According to an embodiment, the subject is a human.

According to an aspect of some embodiments of the present invention, there is provided a cannabinoid composition comprising at least one cannabinoid, a carrier and at least one drug selected from the group consisting of an anticholinergic drug, a beta-3 agonist drug, a botulinum toxic drug, an adrenergic drug, and combinations thereof.

According to an embodiment, said at least one cannabinoid comprises one cannabinoid, two cannabinoids, three cannabinoids, four cannabinoids or more than four cannabinoids.

As used herein, said at least one cannabinoid is selected from the group consisting of tetrahydrocannabiniol (THC), tetrahydrocannabiniolic acid (THCa), cannabidiol (CBD), cannabidiolic acid (CBDa), cannabigerol (CBG), cannabigerolic acid (CBGa), cannabinol (CBN), cannabinolic acid (CBNa), cannabichromene (CBC), cannabichromenic acid (CBCa), cannabicyclol (CBL), cannabicycol acid (CBLa), tetrahydrocannabivarin (THCV), tetrahydrocannabivarin acid (THCVa), cannabidivarin (CBDV), cannabigerovarin acid (CBDVa) and combinations thereof.

According to an embodiment, the carrier is selected from the group consisting of vegetable oils, water, and aqueous solutions.

According to some such embodiments, the composition comprises an anticholinergic drug. According to some such embodiments, the composition comprises an adrenergic drug. According to some such embodiments, the composition comprises an anticholinergic drug and an adrenergic drug, such as for example oxybutinine and mirabegron.

According to an embodiment, the anticholinergic drug is an antimuscarinic agent.

According to an embodiment, the composition further comprises a surfactant. Examples of suitable surfactants include, without limitation, anionic surfactants (such as sodium lauryl sulfate and/or docusate sodium); cationic surfactants (such as benzalkonium chloride and/or cetylpyridinium chloride); amphoteric surfactants (such as betaines, sulfobetaine, amino acids or phospholipids); and non-ionic surfactants (such as a surfactant selected from the group consisting of re Polyoxyethylene sorbitan fatty acid esters (Polysorbate, Tween®), Polyoxyethylene 15 hydroxy stearate (Macrogol 15 hydroxy stearate, Solutol HS15®), Polyoxyethylene castor oil derivatives (Cremophor® EL, ELP, RH 40), Polyoxyethylene stearates (Myrj®), Sorbitan fatty acid esters (Span®), Polyoxyethylene alkyl ethers (Brij®), and Polyoxyethylene nonylphenol ether (Nonoxynol® and combinations thereof).

According to an embodiment, the composition is an oil-in-water emulsion.

According to an aspect of some embodiments of the present invention, there is provided the composition as disclosed herein for use in treating a bladder condition.

According to an embodiment, said treating comprises administration selected from the group consisting of intravesical administration, rectal administration, sublingual administration, and vaginal administration.

According to an embodiment, said administration is intravesical administration. According to some such embodiments, administration is achieved using a device, such as a catheter, that brings said cannabinoids in contact with bladder internal wall.

According to an embodiment, treating further comprises emptying the subject's bladder prior to said administration.

According to an embodiment, the subject is a human.

### EXAMPLES

Examples 1-8

| | | Canna- | | |
|---|---|---|---|---|
| | | binoid | | |
| | | concen- | Terpenes | |
| | Adminis- | tration | concentration | |
| | tration | (% wt) | (% wt) | For treating |
| 1 | Gel | CBD | Myrcene (0.5), | Neurogenic |
| | capsule | (20) | caryophyllene (0.5), | bladder |
| | | | humulene (0.5) | |
| 2 | Sublingual | CBD | Myrcene (0.3), linalool | Overactive |
| | extract | (10):THC | (0.25), limonene | bladder |
| | | (10) | (0.25), nerolidol (0.3), | |
| | | | caryophyllene (0.25) | |
| 3 | Tablets | CBD | Nerolidol (0.45), | Chronic |
| | | (10):THC | borneol (0.2), | pelvic pain |
| | | (5) | humulene (0.2) | syndrome |
| | | | | (CPPS) |
| 4 | Sublingual | CBD | Terpineol (0.15), | Hemorrhagic |
| | extract | (5):THC | linalool (0.15), pinene | cystitis |
| | | (10) | (0.5), myrcene (0.3), | |
| | | | caryophyllene (0.3), | |
| | | | eucalyptol (0.2) | |
| 5 | Gel | CBD | Caryophyllene (0.5), | Cyclophos- |
| | capsule | (5):THC | pinene (0.5), bisabolol | phamide |
| | | (1) | (0.5), myrcene (0.5) | induces |
| | | | | cystitis |
| 6 | Tablet | CBD | Myrcene (0.4), | Interstitial |
| | | (1):THC | caryophyllene (0.4), | cystitis |
| | | (5) | bisabolol (0.2) | |
| 7 | Intravesical | CBD | Caryophyllene (0.5), | Neurogenic |
| | adminis- | (24) | humulene (0.5), pinene | bladder |
| | tration | | (0.5), linalool (0.25) | |
| 8 | Sublingual | CBD | Myrcene (0.8), linalool | Overactive |
| | extract | (32) | (0.8), limonene (0.4) | bladder |

### Example 9: Treating a Man with an Overactive Bladder

A man aged 72 diagnosed with an overactive bladder is treated by administering cannabinoid extract comprising 12% wt THC, 12% wt CBD and terpene blend comprising sabinene, caryophyllene and pinene at a total concentration of 2% wt. Administrating involves 4 droplets per dose, three times per day. Each administrated dose contains 15 mg of THC, 15 mg of CBD and total terpenes of 2.5 mg.

### Example 10: Treating a Man with an Interstitial Cystitis

A man aged 63 diagnosed with an interstitial cystitis is treated by administering cannabinoid gel capsules. Each gel capsule comprises 19.2 mg THC, 3.8 mg CBD and a terpene blend comprising caryophyllene and limonene at a total amount of 2 mg. Three gel capsules are administrated per day.

### Example 11: Treating a Woman with a Neurogenic Bladder

A woman aged 57 diagnosed with a neurogenic bladder is treated by administering cannabinoid extract via an intravesical administration. Administration is achieved using a catheter configured to drain the bladder and apply the medication into the bladder under sterile conditions, thereby bringing said cannabinoids in contact with bladder internal wall. Cannabinoid extract comprises 25% wt THC and 10% wt CBD. Administration involves 30 mg cannabinoid extract twice a day, containing 7.5 mg THC and 3 mg CBD.

### Example 12: Treating a Woman with an Over-Active Bladder

A woman aged 48 diagnosed with an overactive bladder is treated by administering cannabinoid extract comprising 5% wt THC, 5% wt CBD and terpene blend comprising myrcene, linalool, limonene and caryophyllene at a total concentration of 2% wt. Administrating involves 8 droplets per dose, three times per day. Each administrated dose contains 13 mg of THC, 13 mg of CBD and total terpenes of 5 mg. An anticholinergic drug, comprising oxybutynin tablets of 5 mg is administrated simultaneously with the cannabinoid extract.

### Example 13: Treating a Man with Neurogenic Bladder

A man aged 59 diagnosed with neurogenic bladder is treated by administering cannabinoid tablets comprising 76.8 mg CBD and terpene blend comprising nerolidol, geraniol and caryophyllene at a total concentration of 16 mg. Three cannabinoid tablets are administrated per day. Treatment also involves the injection of a 100 unit Botulinumtoxin to the bladder, once a week.

### Example 14: Treating a Woman with a Chronic Pelvic Pain Syndrome

A woman aged 35 diagnosed with Chronic pelvic pain syndrome is treated by administering cannabinoids tablets comprising 4.8 mg THC, 24 mg CBD and terpene blend comprising linalool, myrcene and eucalyptol at a total concentration of 8 mg. Two cannabinoid tablets are administrated per day. Administration of cannabinoids tablets is accompanied by administration of 1 tablet of 2 mg Tolterodine 2 mg.

1-26. (canceled)

27. A method for treating a bladder condition in a subject in need thereof, the method comprising administering to the subject a therapeutically effective dose of a composition comprising at least one cannabinoid and a carrier.

28. The method of claim 27, wherein said bladder condition is a bladder dysfunction, optionally selected from the group consisting of a neurogenic bladder dysfunction, an idiopathic bladder dysfunction, an inflectional bladder dysfunction, an overactive bladder, and combinations thereof.

29. The method of claim 27, wherein said bladder condition is selected from the group consisting of interstitial cystitis, hemorrhagic cystitis, radiation cystitis, cyclophosphamide-induced cystitis, chronic prostatitis/chronic pelvic pain syndrome (CPPS) and combinations thereof.

30. The method of claim 27, wherein said at least one cannabinoid is selected from the group consisting of tetrahydrocannabiniol (THC), tetrahydrocannabiniolic acid (THCa), cannabidiol (CBD), cannabidiolic acid (CBDa) and combinations thereof.

31. The method of claim 27, wherein said at least one cannabinoid comprises at least one selected from the group consisting of THC and THCa and at last one selected from the group consisting of CBD and CBDa, optionally wherein a (total THC+THCa) to (total CBD+CBDa) mol/mol ratio is between 5:1 and 1:5 or wherein a (total THC+THCa) to (total CBD+CBDa) mol/mol ratio is less than 0.1:1.

32. The method of claim 27, wherein said at least one cannabinoid is present at a concentration of from about 1% to about 35% w/w of the total composition.

33. The method of claim 27, wherein said composition further comprises at least one terpene selected from the group consisting of pinene, limonene, linalool, caryophyllene, myrcene, humulene, terpineol, nerolidol, bisabolol, geraniol, eucalyptol, borneol, sabinene, terpinolene, ocimene and combinations thereof.

34. The method of claim 33, wherein a total terpenes to total cannabinoids weight/weight ratio in said composition is at least 0.05:1.

35. The method of claim 27, further comprising administering to said subject a therapeutically effective dose of at least one drug selected from the group consisting of an anticholinergic drug, a beta-3 agonist drug, a botulinum toxic drug, an adrenergic drug, and combinations thereof.

36. The method of claim 35, wherein said at least one drug is selected from the group consisting of Oxybutinin, Tolterodine, Trospium, Fesoterodine (Toviaz), Mirabegron (Myrbetriq), Botulinumtoxin A and combinations thereof.

37. The method of claim 27, wherein said composition further comprises at least one drug selected from the group consisting of an anticholinergic drug, a beta-3 agonist drug, a botulinum toxic drug, an adrenergic drug, and combinations thereof.

38. The method of claim 27, wherein said composition is an oil-in-water emulsion.

39. The method of claim 27, wherein said cannabinoid is present in said dose in an amount of between 1 mg and 100 mg.

40. The method of claim 27, wherein said administering is selected from the group consisting of intravesical administration, rectal administration, oral administration, nasal administration, sublingual administration, vaginal administration, intravesical administration and inhalation.

41. The method of claim 40, wherein said intravesical administration uses a device that brings said cannabinoids in contact with bladder internal wall.

42. The method of claim 27, further comprising emptying the subject's bladder prior to said administering.

43. A cannabinoid composition comprising at least one cannabinoid, a carrier and at least one drug selected from the group consisting of an anticholinergic drug, a beta-3 agonist drug, a botulinum toxic drug, an adrenergic drug, and combinations thereof.

44. The cannabinoid composition of claim 43, further comprising a surfactant.

45. The cannabis composition of claim 43, wherein said composition is an oil-in-water emulsion.

46. A method of treating a bladder condition in a subject in need thereof, the method comprising administering to the subject a therapeutically effective dose of the cannabinoid composition of claim 43
